(19) United States
(12) Patent Application Publication
(10) Pub. No.: US 2026/0259163 A1
(43) Pub. Date: Sep. 3, 2026

(54) MEASUREMENT APPARATUS AND MEASUREMENT METHOD

(71) Applicant: SONY GROUP CORPORATION, TOKYO (JP)

(72) Inventors: MITSUHIRO KAWANISHI, TOKYO (JP); YUICHI ISHIDA, TOKYO (JP); TAIKI SUGIYAMA, TOKYO (JP); MICHIKO NAKAO, TOKYO (JP); KAZUHIKO MIYAHARA, TOKYO (JP); RYOTA YAMANO, TOKYO (JP)

(21) Appl. No.: 18/858,409

(22) PCT Filed: Mar. 9, 2023
(86) PCT No.: PCT/JP2023/008975

§ 371 Date: Oct. 21, 2024

(30) Foreign Application Priority Data
Apr. 28, 2022 (JP) 2022-074906

(51) Int. Cl. G01N 27/12; G01N 27/14; G01N 33/00

(52) U.S. Cl. CPC G01N 27/124; G01N 27/14; G01N 33/0029; G01N 33/0047

(57) ABSTRACT

[Object] To provide a measurement apparatus and a measurement method that are capable of easily identifying a specific gas species.

[Solving Means] A measurement apparatus according to an embodiment of the present technology includes: a measurement unit; a measurement unit; a gas supply unit; and a control unit.

The measurement unit includes a metal oxide semiconductor sensor and a heating unit that heats the semiconductor sensor to a first temperature, a second temperature, or a third temperature, the second temperature and the third temperature being higher than the first temperature.

The control unit controls the measurement unit and the gas supply unit.

The control unit is capable of executing
refresh treatment for heating the semiconductor sensor in a first gas atmosphere that does not contain a reducing gas to the third temperature to clean the semiconductor sensor,
low-temperature measurement treatment for heating the semiconductor sensor to the first temperature in a second gas atmosphere that contains the reducing gas to measure a resistance value of the semiconductor sensor, and
high-temperature measurement treatment for heating the semiconductor sensor to the second temperature in the second gas atmosphere to measure the resistance value of the semiconductor sensor, and
the control unit executes the low-temperature measurement treatment as a process following the refresh treatment.

### TECHNICAL FIELD

The present technology relates to a measurement apparatus and a measurement method to be used for detecting and identifying a gas.

### BACKGROUND ART

A technology for measuring a resistance value of a metal oxide (MOx) semiconductor sensor in each of a high-temperature range and a low-temperature range in order to detect and identify a gas has been known. For example, Patent Literature discloses a gas detection apparatus that periodically and alternately changes the heating temperature of a gas sensor that uses a metal oxide semiconductor whose resistance value changes depending on the gas between a high-temperature range (400° C.) and a low-temperature range (80° C.), and includes: a combustible gas detection means for detecting from the output of the gas sensor in the high-temperature range; and a preliminary detection means for preliminary detecting carbon monoxide from the output of the gas sensor in the low-temperature range.

### CITATION LIST

### Patent Literature

Patent Literature 1: Japanese Patent Application Laid-open No. 1987-223662

### DISCLOSURE OF INVENTION

### Technical Problem

By spraying a reducing gas while heating the MOx semiconductor sensor, it is possible to detect a gas from the amount of change (sensitivity) in the resistance value of the semiconductor sensor. However, in Patent Literature 1, since moisture and impurity gases are present in the atmosphere measured in the low-temperature range, the output of the gas sensor in the low-temperature range becomes low or the output value varies widely in some cases. For this reason, it has been difficult to identify the type of gas using the output of the gas sensor in the low-temperature range.

In view of the circumstances as described above, it is an object of the present technology to provide a measurement apparatus and a measurement method that are capable of easily identifying a specific gas species.

### Solution to Problem

A measurement apparatus according to an embodiment of the present technology includes: a measurement unit; a gas supply unit; and a control unit.

The measurement unit includes one or more semiconductor sensors each including an adsorption layer formed of metal oxide, a measurement chamber that houses the semiconductor sensor, and a heating unit that heats the semiconductor sensor to a first temperature, a second temperature, or a third temperature, the second temperature and the third temperature being higher than the first temperature.

The gas supply unit includes a first gas supply line and a second gas supply line, the first gas supply line supplying a first gas that does not contain a reducing gas to be detected to the measurement chamber, the second gas supply line supplying a second gas that contains the reducing gas to the measurement chamber.

The control unit controls the measurement unit and the gas supply unit.

The control unit is capable of executing
refresh treatment for cleaning the adsorption layer while heating the semiconductor sensor to the third temperature in an atmosphere of the first gas,
low-temperature measurement treatment for measuring a resistance value of the semiconductor sensor while heating the semiconductor sensor to the first temperature in an atmosphere of the second gas, and
high-temperature measurement treatment for measuring the resistance value of the semiconductor sensor while heating the semiconductor sensor to the second temperature in the atmosphere of the second gas,
the control unit executing the low-temperature measurement treatment as a process following the refresh treatment.

The control unit may execute
low-temperature measurement pre-treatment for measuring the resistance value of the semiconductor sensor while heating the semiconductor sensor to the first temperature in the atmosphere of the first gas as a process between the refresh treatment and the low-temperature measurement treatment, and
high-temperature measurement pre-treatment for measuring the resistance value of the semiconductor sensor while heating the semiconductor sensor to the second temperature in the atmosphere of the first gas as a process before the high-temperature measurement treatment, and calculate
a first sensitivity that is a ratio of the resistance value measured in the low-temperature measurement pre-treatment to the resistance value measured in the low-temperature measurement treatment, and
a second sensitivity that is a ratio of the resistance value measured in the high-temperature measurement pre-treatment to the resistance value measured in the high-temperature measurement treatment.

The first temperature may be 25° C. or more and 200° C. or less, and the second temperature may be 200° C. or more and 500° C. or less.

The third temperature may be 300° C. or more and 600° C. or less.

An air atmosphere in which the reducing gas is not present may be dry air with a relative humidity of 10% or less.

The semiconductor sensor may include a first semiconductor sensor for the low-temperature measurement treatment and a second semiconductor sensor for the high-temperature measurement treatment, and the control unit may simultaneously execute the low-temperature measurement treatment and the high-temperature measurement treatment.

The second gas supply line may include a branch line that branches from the first gas supply line and a supply source of the reducing gas disposed in the branch line, and supply the second gas to the measurement chamber as a mixed gas of the first gas and the reducing gas.

The first gas supply line may include a first passage that includes a first filter for collecting moisture and the reducing gas, a second passage that includes a second filter for collecting the moisture and the reducing gas, and a switching unit that selectively switches between the first passage and the second passage.

The first gas supply line may further include a heater that is capable of heating the first filter and an exhaust passage for discharging moisture desorbed from the first filter by the heater.

The measurement apparatus may further include an analysis unit that include a determination unit that determines a type of the reducing gas on the basis of the first sensitivity and the second sensitivity calculated by the control unit.

The determination unit may determine whether or not the reducing gas contains a hydrocarbon substance emitted from a living thing such as a plant, an animal, and a human.

The hydrocarbon substance may be a substance that contains a terpene organic compound.

A measurement method according to an embodiment of the present technology includes: controlling, by a control unit, a measurement unit that includes one or more semiconductor sensors each including an adsorption layer formed of metal oxide, a measurement chamber that houses the semiconductor sensor, and a heating unit that heats the semiconductor sensor to a first temperature, a second temperature, or a third temperature, the second temperature and the third temperature being higher than the first temperature, and a gas supply unit that includes a first gas supply line and a second gas supply line, the first gas supply line supplying a first gas that does not contain a reducing gas to be detected to the measurement chamber, the second gas supply line supplying a second gas that contains the reducing gas to the measurement chamber.

The control unit executes,
refresh treatment for cleaning the adsorption layer while heating the semiconductor sensor to the third temperature in an atmosphere of the first gas,
low-temperature measurement treatment for measuring a resistance value of the semiconductor sensor while heating the semiconductor sensor to the first temperature in an atmosphere of the second gas, and
high-temperature measurement treatment for measuring the resistance value of the semiconductor sensor while heating the semiconductor sensor to the second temperature in the atmosphere of the second gas, and
executes the low-temperature measurement treatment as a process following the refresh treatment.

### BRIEF DESCRIPTION OF DRAWINGS

FIG. 1 is a schematic cross-sectional view showing a configuration of a main part of a semiconductor sensor to be used in a first embodiment of the present technology.

FIG. 2 is a drive circuit of the semiconductor sensor.

FIG. 3 is a diagram showing an example of the change over time in the resistance value output from the semiconductor sensor.

FIG. 4 is a schematic diagram showing a relationship between the heating temperature and the resistance value of the semiconductor sensor.

FIG. 5 is a diagram showing the temperature and sensitivity properties of various organic molecules in an existing method of use.

FIG. 6 is a diagram showing sensor resistance values at a low temperature in an existing method of use.

FIG. 7 is a schematic configuration diagram of a measurement system.

FIG. 8 is a diagram showing the change in the resistance value of the semiconductor sensor 10 during thermal refresh.

FIG. 9 is a graph when an evaluation gas is supplied to the semiconductor sensor 10 at 100° C. after the thermal refresh, where Part (A) is a diagram showing the change in the resistance value and Part (B) is a diagram showing the sensitivity properties.

FIG. 10 is a flowchart showing an example of a procedure for measuring the change in the resistance value.

FIG. 11 is a schematic configuration diagram of a measurement apparatus according to a second embodiment.

FIG. 12 is a diagram plotting main component scores, where Part (A) is a diagram plotting main component scores of gases to be identified, which were calculated in advance, Part (B) is a diagram showing the main component analysis of an unknown gas and prior data, and Part (c) is a diagram in which data regions of gases to be identified overlap with each other.

FIG. 13 is a diagram plotted on the basis of main component scores, where Part (A) is a diagram plotting only high-temperature data and Part (B) is a diagram plotting high-temperature data and low-temperature data.

FIG. 14 is a schematic configuration diagram of a semiconductor sensor according to a modified example 1 that includes a low-temperature driving sensor and a high-temperature driving sensor.

FIG. 15 is a schematic configuration diagram of a gas supply unit in a modified example 2.

FIG. 16 is a diagram showing a control flow of a collection tube and a control flow of a semiconductor sensor.

FIG. 17 is a schematic configuration diagram obtained by replacing a pump unit in the modified example 2 with a filter and omitting a permeator.

FIG. 18 shows a modified example of the filter in FIG. 17, where Part (A) is a schematic configuration diagram showing of a gas supply unit including a regenerative filter that can be regenerated by combining a plurality of filters and is capable of constantly supplying air from which moisture and the like have been removed, and Part (B) is a schematic configuration diagram of a gas supply unit in which the filter includes a plurality of filters.

FIG. 19 is a schematic configuration diagram of a gas supply unit that supplies a first gas and a second gas to a measurement chamber through the filter.

### MODE(S) FOR CARRYING OUT THE INVENTION

Embodiments according to the present technology will be described below with reference to the drawings.

### First Embodiment

### [Semiconductor Sensor]

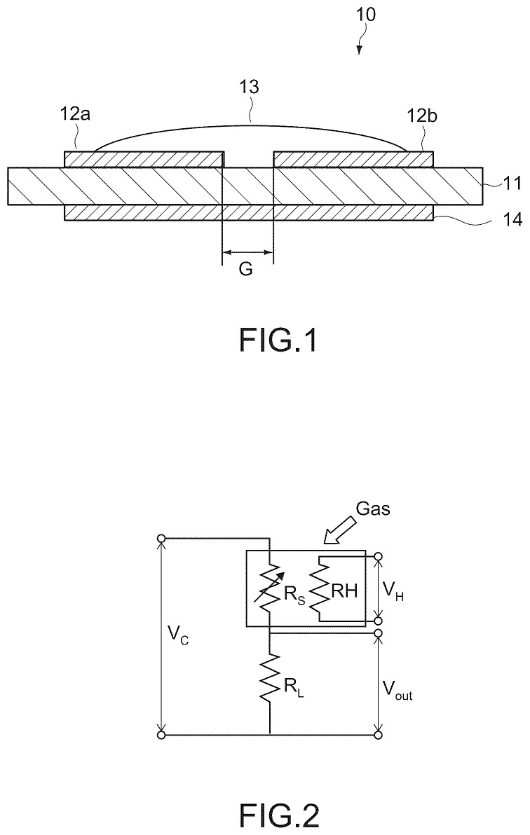

FIG. 1 is a schematic cross-sectional view showing a configuration of a main part of a semiconductor sensor 10 to be used in a first embodiment of the present technology.

The semiconductor sensor 10 includes a substrate 11, a pair of electrodes 12a and 12b formed on the surface of the substrate 11, an adsorption layer 13 provided between the pair of electrodes 12a and 12b, and a heating layer 14 disposed between the back surface of the substrate 11.

The substrate 11 is, for example, an alumina substrate, a silicon substrate, or a quartz substrate, and the pair of electrodes 12a and 12b are formed of, for example, metal layers such as Ti, Au, Pt, or stacked films thereof, and formed on the surface of the substrate 11 with a gap G therebetween.

The adsorption layer 13 is formed of a metal oxide (MOx) that includes a sintered body containing a metal oxide material and a catalyst metal material. Examples of the metal oxide material include a tungsten oxide, an indium oxide, a tin oxide, and a zinc oxide. Examples of the catalyst metal material include iridium, an oxide thereof, palladium, an oxide thereof, rhenium, an oxide thereof, platinum, and gold.

The heating layer 14 is for heating the adsorption layer 13 to a predetermined temperature, and includes, for example, a ceramic heater or a platinum heater. The heating layer 14 is connected to a heater power source (not shown), and is configured such that it can be heated to, for example, a temperature of 600° C. or less by a control device described below.

In general, when spraying a reducing gas while heating a MOx semiconductor sensor, the resistance value of the semiconductor sensor begins to change from a certain temperature. For this reason, a gas can be detected or quantified from the amount of change (sensitivity) of the resistance value. Further, in the case where there is a large difference between the temperature and the profile of the amount of change (sensitivity) of the resistance, the type of gas can be identified.

FIG. 2 is a drive circuit of the semiconductor sensor 10. In the figure, Rs represents a sensor resistor, RL represents a load resistor connected to the sensor resistor Rs in series, RH represents a heater resistor that heats the semiconductor sensor 10, and the heating temperature of the heating layer 14 is adjusted by a heater voltage VH. Then, the sensor resistor Rs can be measured by the following formula on the basis of a voltage Vout across the load resistor RL when a power supply voltage Vc is applied across the series resistors (Rs and RL).

$$Rs = ( ( Vc - Vout ) / Vout ) \times RL$$

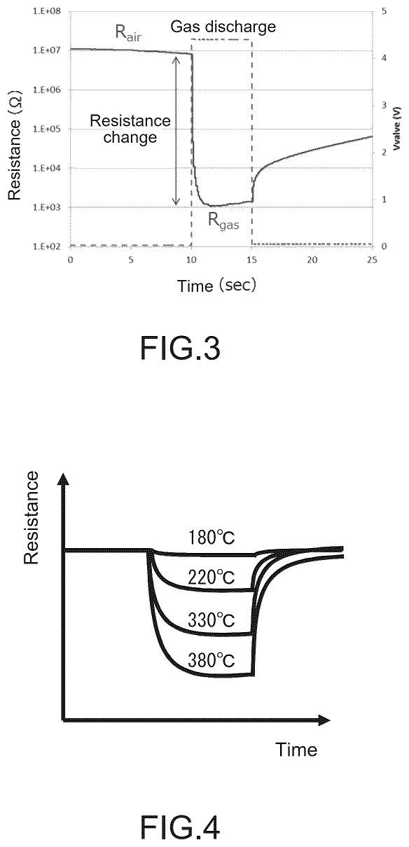

FIG. 3 shows an example of the change over time in the resistance value Rs output from the semiconductor sensor 10. In this example, the semiconductor sensor 10 was driven in an air atmosphere in which a reducing gas to be detected was not present, a reducing gas to be detected was discharged into the air atmosphere for an arbitrary time (5 seconds in this example).

Note that the air (first gas) atmosphere in which a reducing gas to be detected is not present represents a state in which a specific substance is managed to be removed by passing through a filter such as activated carbon, silica gel, and molecular sieve by a pump. The above specific substance is, for example, water vapor or a VOC (volatile organic compound) in the environment. Here, a first gas can take not only the state in which the reducing gas is completely removed, but also any state in which the reducing gas is managed to be removed by a filter. That is, the first gas includes a state in which the reducing gas is not completely removed by the filter and only a small amount of the reducing gas is present.

As shown in FIG. 3, a resistance value Rgas of the semiconductor sensor 10 in the air (second gas) atmosphere in which a reducing gas to be detected is present is lower than a resistance value Rair of the semiconductor sensor 10 in the clean air atmosphere. When the discharge of the reducing gas is stopped, the output of the semiconductor sensor 10 returns to the resistance value (Rair) in the clean air atmosphere.

The greater the decrease in Rgas relative to Rair, the greater the sensitivity to the reducing gas. In the following description, the ratio of Rair to Rgas (Rair/Rgas) is defined as the sensor sensitivity.

The resistance value and sensitivity of the semiconductor sensor 10 change depending on the heating temperature of the heating layer 14. FIG. 4 is a schematic diagram showing a relationship between the heating temperature and the resistance value (hereinafter, referred to also as a resistance value profile), and shows that the higher the heating temperature, the lower resistance value.

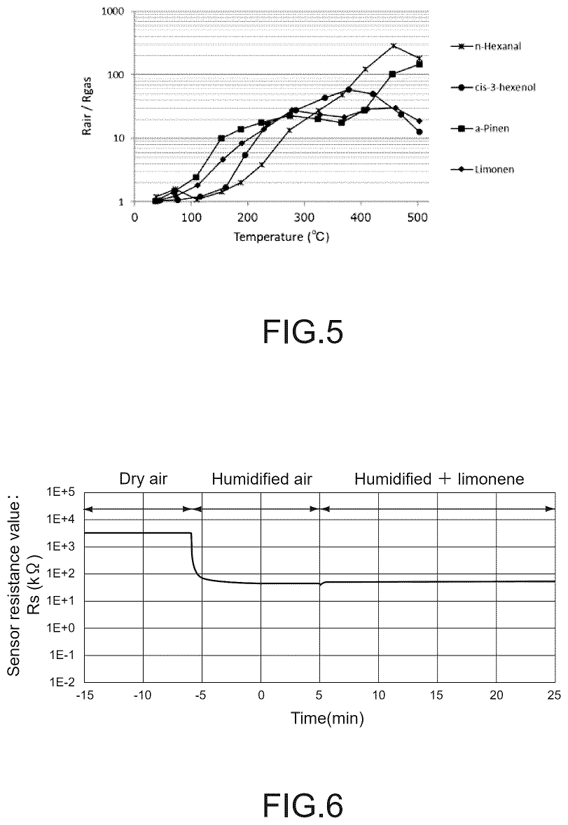

FIG. 5 is a diagram showing the temperature and sensitivity properties of various organic molecules in an existing method of use. As shown in FIG. 5, the gas detection of the semiconductor sensor 10 has sensitivity peaks near high temperatures (200° C. or more and 500° C. or less), and the sensitivity is calculated using the temperature as the drive temperature. In the existing method of use, the sensitivity decreases as the temperature is lowered, and only low sensitivity is obtained at approximately 200° C. or less, which has been considered ineffective for use in identification.

The reason why high sensitivity cannot be obtained at low temperatures (25° C. or more and 200° C. or less) is presumably that humidity and impurity gases are present in an actual environment, they are adsorbed onto the surface of the sensitive film, and the difference between the reference resistance value (corresponding to Rair) and the resistance value (corresponding to Rgas) of an evaluation gas (reducing gas to be detected) becomes smaller.

FIG. 6 is a diagram showing sensor resistance values at a low temperature in an existing method of use. Here, as the electrodes 12a and 12b of the semiconductor sensor 10, comb-shaped Ti/Pt films having a thickness of 500 nm are placed with a gap of 50 μm on the substrate 11 that has a thickness of 400 μm and is formed of alumina (see FIG. 1). A drive voltage Vc (see FIG. 2) input between the electrodes 12a and 12b was set to 5 V. The adsorption layer 13 was an n-type metal oxide semiconductor (SnO2) that contains a tin oxide as a metal oxide material and had a thickness of 50 μm. The heating temperature by the heater was set to 100° C.

As shown in FIG. 6, for example, it has been shown that the resistance value decreases significantly by changing from dry air to humidified air at 100° C., (reproducing the humidity in an actual environment) and a change in the resistance value is not obtained (sensitivity is not obtained) even if +limonene that is an evaluation gas is introduced. This is presumably because the adsorption and retention of water molecules to the surface of the sensitive film inhibit the adsorption of evaluation gas molecules. Further, it is conceivable that adsorption and retention of gas molecules present in the atmosphere other than water molecules to the surface of the sensitive film also inhibit the adsorption of evaluation gas molecules.

That is, in the existing method of use, the sensitivity in the low-temperature driving is low and it is difficult to identify the type of gas. Further, even if the volatile organic compound is identified only in the high-temperature driving, it is difficult to identify reducing gases having similar structures.

Meanwhile, the present inventors have found by the experiment shown below that high sensitivity can be achieved even in the low-temperature driving (see Table 1). That is, regarding the sensitivity, the sensitivity at low temperatures (25° C. or more and 200° C. or less) is higher than the sensitivity at high temperatures (200° C. or more and 500° C. or less). Experimental examples in which high sensitivity was achieved even in the low-temperature driving will be shown below.

### Experimental Example

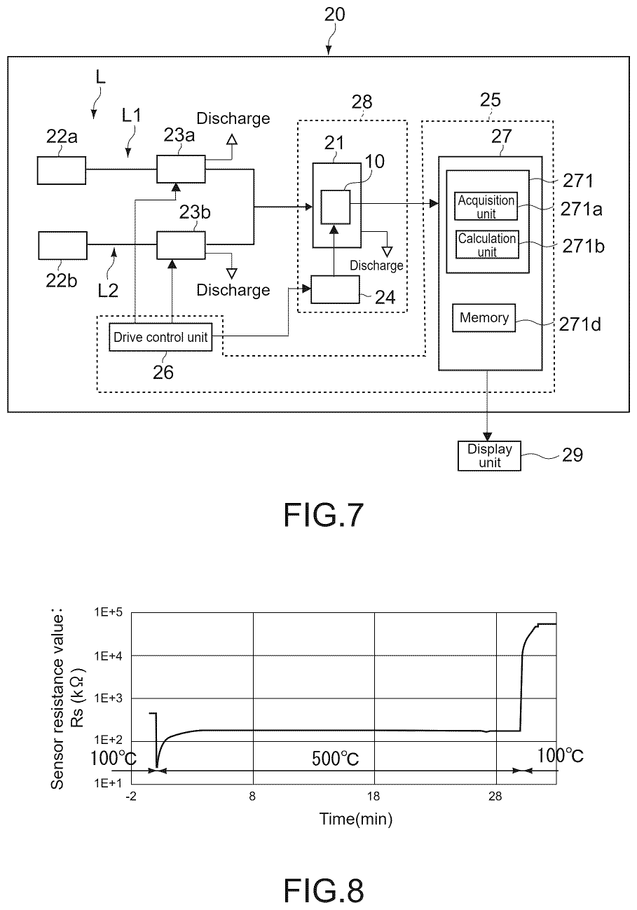

The present inventors measured the change in the resistance value of various reducing gases by the semiconductor sensor 10 using a measurement system 20 shown in FIG. 7. FIG. 7 is a schematic configuration diagram of the measurement system 20, and the respective units will be described.

### [Configuration of Measurement System]

The measurement system 20 includes a measurement unit 28, a gas supply unit L, and a control unit 25. The measurement unit 28 includes one or more semiconductor sensors 10, a measurement chamber 21 that houses the semiconductor sensor 10, and a heating unit 24 that is capable of the semiconductor sensor 10 to a first temperature (25° C. or more and 200° C. or less) that is a low temperature, a second temperature (200° C. or more and 500° C. or less) that is a high temperature, and a third temperature (300° C. or more and 600° C. or less) that is a temperature during refresh treatment described below.

The gas introduced into the measurement chamber 21 is discharged from the measurement chamber 21 in order to maintain the inside of the measurement chamber 21 at a constant pressure.

The gas supply unit L includes a dry air introduction line (first gas supply line) L1 and a reducing gas introduction line (second gas supply line) L2, the dry air introduction line L1 supplying a first gas that does not contain a reducing gas to be detected to the measurement chamber 21, the reducing gas introduction line L2 supplying a second gas that contains the reducing gas to the measurement chamber 21.

The dry air introduction line L1 is configured to be capable of introducing dry air (with a relative humidity of 10% or less. The same applies hereinafter) to the measurement chamber 21 via a three-way valve 23a and a first gas control unit 22a (described below).

The reducing gas introduction line L2 is configured to be capable of introducing an air atmosphere in which a reducing gas to be detected is present into the measurement chamber 21 via a second gas control unit 22b and a three-way valve 23b by driving a permeator (PD-18, GASTEC CORPORATION) (not shown). In this embodiment, the conditions of the permeator were set to 50° C. and 2 L/min using the diffusion tube of D-30. It goes without saying that the conditions are not limited thereto and can be set as appropriate.

As the first gas control unit 22a and the second gas control unit 22b, a pressure valve and a flow meter were used. The three-way valve 23a is an open/close valve that is capable of switching between a state in which the first gas control unit 22a and the measurement chamber 21 communicate with each other and a state in which the first gas control unit 22a and the outside of the measurement system 20 communicate with each other. Meanwhile, the three-way valve 23b is an open/close valve that is capable of switching between a state in which the second gas control unit 22b and the measurement chamber 21 communicate with each other and a state in which the second gas control unit 22b and the outside of the measurement system 20 communicate with each other.

In this embodiment, the first gas and the second gas are constantly supplied to the gas supply unit L. That is, when the first gas is supplied to the measurement chamber 21 through the dry air introduction line L1, the second gas maintains the state of being discharged to the outside of the measurement system 20 by the three-way valve 23b. When the second gas is supplied to the measurement chamber 21 through the reducing gas introduction line L2, the first gas maintains the state of being discharged to the outside of the measurement system 20 by the three-way valve 23a. As a result, it is possible to more stably supply the gas than turning on/off the supply source each time the gas is switched.

The control unit 25 includes a drive control unit 26 and a measurement apparatus 27. The drive control unit 26 controls the opening and closing of the three-way valves 23a and 23b, and the heating unit 24 that heats the heating layer 14 (see FIG. 1) of the semiconductor sensor 10.

The drive control unit 26 causes one of the dry air introduction line L1 and the reducing gas introduction line L2 to communicate with the measurement chamber 21, and causes the other to communicate with the outside of the measurement system 20. For example, in the case where only dry air is desired to be supplied to the measurement chamber 21, the drive control unit 26 controls the three-way valve 23a such that the dry air introduction line L1 and the measurement chamber 21 communicate with each other, and controls the three-way valve 23b such that the reducing gas introduction line L2 and the outside of the measurement system 20 communicate with each other.

Further, the drive control unit 26 controls the heating unit 24 to control the heating temperature of the semiconductor sensor 10. In this embodiment, the heating temperature of the semiconductor sensor 10 is arbitrarily adjusted in the range of 25° C. to 600° C.

The measurement apparatus 27 includes a control unit 271 and a memory 271d, the control unit 271 including an acquisition unit 271a and a calculation unit 271b.

The acquisition unit 271a acquires, on the basis of the output Vout (see FIG. 2) of the semiconductor sensor 10, a resistance value (R1air) of the semiconductor sensor 10 when the semiconductor sensor 10 is heated at the first temperature in the above first gas atmosphere and a resistance value (R1gas) of the semiconductor sensor 10 when the semiconductor sensor 10 is heated at the first temperature in the above second gas atmosphere.

The acquisition unit 271a acquires a resistance value (R2air) of the semiconductor sensor 10 when the semiconductor sensor 10 is heated at the second temperature in the above first gas atmosphere and a resistance value (R2gas) of the semiconductor sensor 10 when the semiconductor sensor 10 is heated at the second temperature in the above second gas atmosphere. The acquisition unit 271a may include a calculation unit that converts the output (Vout) of the semiconductor sensor 10 into the resistance value Rgas.

The calculation unit 271b calculates the change (sensitivity) in the resistance value on the basis of the resistance value acquired by the acquisition unit 271a. Specifically, the first sensitivity in the first temperature is calculated by the calculation unit 271b as (R1air)/(R1gas). The second sensitivity in the second temperature is calculated by the calculation unit 271b as (R2air)/(R2gas).

The memory 271d is an information storage device such as a semiconductor memory and a hard disk. The memory 271d stores a program for causing the acquisition unit 271a and the calculation unit 271b to operate as functional blocks of the CPU 271. Further, the memory 271d stores pieces of data regarding the resistance values ((R1air), (R1gas), (R2air), and (R2gas)) and the sensitivities (the first sensitivity and the second sensitivity) of the semiconductor sensor 10 described above.

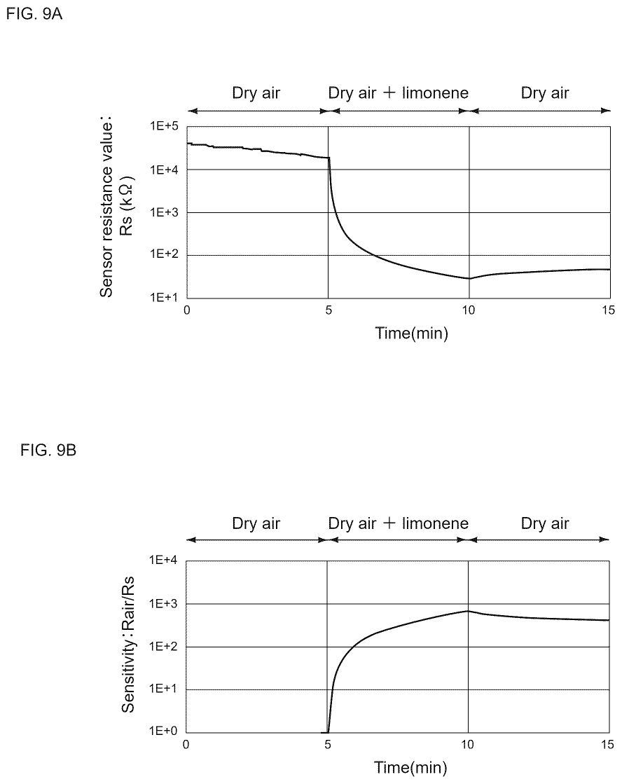

The output of the measurement apparatus 27 may be displayed on a display unit 29. The display unit 29 displays, for example, the resistance value profile and the sensitivity profile of the semiconductor sensor 10 as shown in Parts (A) and (B) of FIG. 9, or Table 1 described below.

Using the measurement system 20 configured as described above, response properties of the semiconductor sensor 10 at a low temperature were evaluated as follows.

### [Experimental Method]

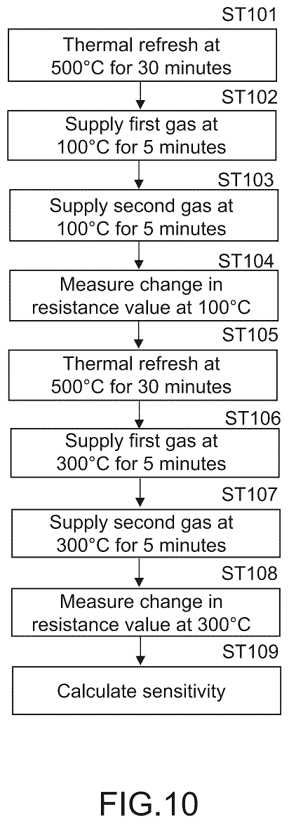

FIG. 8 is a diagram showing the change in the resistance value of the semiconductor sensor 10 during thermal refresh, and FIG. 9 is a graph when an evaluation gas is supplied to the semiconductor sensor 10 at 100° C. after the thermal refresh, where Part (A) is a diagram showing the change in the resistance value and Part (B) is a diagram showing the sensitivity properties. FIG. 10 is a flowchart showing an example of a procedure for measuring the change in the resistance value.

Regarding the measurement conditions shown in FIG. 8, the sensitive film of the semiconductor sensor 10 is formed of SnO₂, and the pump unit 22a is driven to supply dry air with a humidity of 1% or less to the measurement chamber 21 at 2 L/min.

Here, the thermal refresh represents a process performed before measuring the change in the resistance value of the semiconductor sensor 10 at a low temperature (25° C. or more and 200° C. or less, 100° C. in this embodiment) or a high temperature (300° C. or more and 500° C. or less, 300° C. in this embodiment), and moisture and impurity molecules adsorbed to the semiconductor sensor 10 are removed by heating the semiconductor sensor 10 at the high temperature. More specifically, it is a process for controlling the heating unit 24 by the drive control unit 26 to heat the semiconductor sensor 10 at the third temperature (300° C. or more and 600° C. or less, 500° C. in this embodiment) for 30 minutes.

As shown in FIG. 8, first, while dry air that is the first gas is supplied from the first gas control unit 22a to the semiconductor sensor 10 heated to 100° C., the heating unit 24 is controlled by the drive control unit 26 to increase the temperature of the semiconductor sensor 10 to 500° C. (Step 101).

After the thermal refresh process, the drive control unit 26 performs control such that the temperature of the semiconductor sensor 10 is 100° C. and dry air is supplied to the measurement chamber 21 for 5 minutes to stabilize the resistance value (Step 102).

Next, after supplying dry air to the measurement chamber 21 for 5 minutes, the three-way valves 23a and 23b are switched to supply dry air containing evaporated limonene, which is the second gas, from the permeator to the measurement chamber 21 at 2 L/min for 5 minutes (Step 103).

After supplying for 5 minutes, the three-way valves 23a and 23b are switched to supply dry air from the first gas control unit 22a to the measurement chamber 21.

The resistance value (R1air) immediately before supplying +limonene and the resistance value (R1gas) immediately before supplying +limonene for 5 minutes and switching to only dry air are measured to calculate the change in the resistance value (first sensitivity) (see Parts (A) and (B) of FIG. 9) (Step 104).

When measuring the change in the resistance value (second sensitivity) of the semiconductor sensor 10 at the high temperature after measuring the sensitivity of the semiconductor sensor 10 at the low temperature, the above-mentioned thermal refresh process is performed in the same manner (Step 105), and then, dry air is supplied to the measurement chamber 21 at 300° C. for 5 minutes (Step 106).

Next, after supplying dry air to the measurement chamber 21 for 5 minutes, the three-way valves 23a and 23b are switched to supply dry air containing evaporated limonene from the permeator to the measurement chamber 21 at 2 L/min for 5 minutes (Step 107).

After supplying for 5 minutes, the three-way valves 23a and 23b are switched to supply dry air from the first gas control unit 22a to the measurement chamber 21.

The resistance value (R2air) immediately before supplying +limonene and the resistance value (R2gas) immediately before supplying +limonene for 5 minutes and switching to only dry air are measured to calculate the change in the resistance value (second sensitivity) (see Parts (A) and (B) of FIG. 9) (Step 108).

The calculation unit 271b calculates the sensitivity on the basis of the resistance values measured in Step 104 and Step 108 (Step 109).

In the case of measuring at a high temperature, the thermal refresh process (Step 105) may be omitted. Further, although the humidity of the dry air was set to 1% or less in this experimental method, the humidity is not limited thereto and only needs to be 10% or less.

### [Experimental Results]

Table 1 shows the results of the second sensitivity at a high temperature (300° C.) and the first sensitivity at a low temperature (100° C.) measured by the above experimental method by a combination of +limonene and SnO₂ as well as other combinations of reducing gases and sensitive films.

TABLE 1

| | High-temperature driving (300° C.) | | | | Low-temperature driving (100° C.) | | | |
|---|---|---|---|---|---|---|---|---|
| | In2O3 | SnO2 | WO3 | ZnO | In2O3 | SnO2 | WO3 | ZnO |
| Limonen | 5.2 | 5 | 17.8 | 55.4 | 6633.1 | 357.7 | 882.3 | 7120.2 |
| a-Pinen | 8.6 | 15.3 | 27.1 | 88.9 | 17524 | 1081.8 | 2810.5 | 18651.6 |
| cis-3-hexenol | 8 | 6.5 | 8.2 | 46.5 | 7.2 | 5 | 11.9 | 2.1 |
| n-Hexanal | 93.7 | 17.9 | 17.7 | 88.8 | 632.4 | 356.7 | 14.2 | 316.3 |
| trans-2-Hexenal | 59.8 | 13.2 | 19.8 | 38 | 271.3 | 222.3 | 5.4 | 878.4 |

As shown in Table 1, which shows the experimental results, it can be seen that s sensitivity higher than the sensitivity during high-temperature driving can be achieved by a combination of a specific sensitive film material and a reducing gas in the low-temperature driving. It can be seen that the sensitivity at a low temperature is greatly improved for particularly a terpene organic compound such as an α-pinene and limonene although the sensitivity differs depending on the type of sensitive film.

The reason why the sensitivity of the semiconductor sensor 10 at the low temperature is higher than the sensitivity of the semiconductor sensor 10 at the high temperature is presumably as follows.

That is, it is conceivable that impurity molecules and water molecules that are present on the surface of the sensitive film are removed by the thermal refresh process before measuring the sensitivity of the semiconductor sensor 10, the resistance value (R1air), which is a base at the time of the low-temperature, can be significantly improved, and high sensitivity properties are achieved.

As a result, since not only the sensitivity properties at the high temperature but also the sensitivity properties at the low temperature can be used, it is possible to more easily identify a reducing gas to be detected.

For example, assumption is made that the sensitivity of a certain gas is measured using a sensitive film In₂O₃, and a value of 8.3 at the high temperature (300° C.) and a value of 17500 at the low temperature are calculated. In the existing measurement, measurement has to be performed only at the high temperature because no sensitivity can be achieved at the low temperature. For this reason, it has been difficult to distinguish between α-pinene and cis-3-hexenol with only the value of 8.3. However, as in this embodiment, by improving the sensitivity at the low temperature, it is possible to identify the above certain gas as α-pinene.

Examples of an application example of the above semiconductor sensor 10 include a detection apparatus having a function of detecting damage caused by pests in plants as described below.

The above-mentioned measurement method is applicable to detect damage caused by pests in plants when cultivating plants in the entire agricultural industry including plant factories.

It is known that when plants are damaged by pests, the plants release volatile molecules called green leaf volatiles (GLVs). The green leaf volatiles represent the main component of the green leaf scent and grassy smell, and approximately nine types, i.e., (Z)-3-hexenal, (Z)-3-hexenol, (Z)-3-hexenyl acetate, (E)-2-hexenal, (E)-2-hexenol, (E)-2-hexenyl acetate, n-hexanal, n-hexanol, n-hexanyl acetate, are currently known. In addition, it is known that plants release volatile chemical substances that attract natural enemies (such as a parasitic wasp) of the herbivore, such as herbivore-induced plant volatiles (HIPVs), which are specifically produced when the plants are damaged by pests or the like. Terpenes and terpenoids are common in this category and characterized by releasing a unique blend of odors depending on the type of pest, and xx-pinene, d-limonene, (Z)-β-ocimene, jasmonic acid, and the like are known.

### Second Embodiment

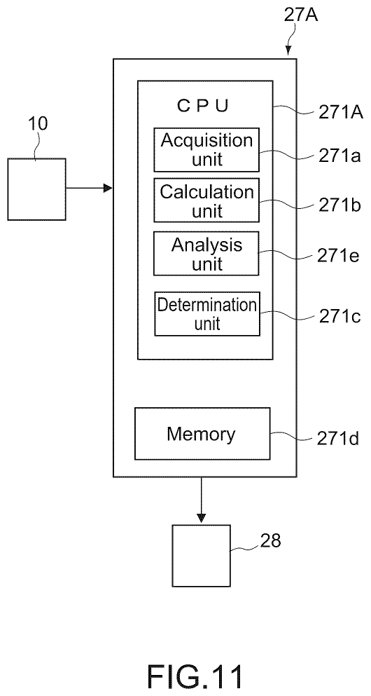

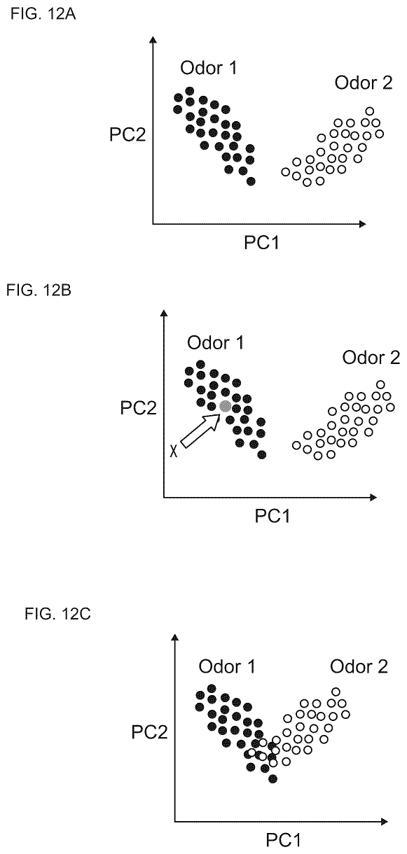

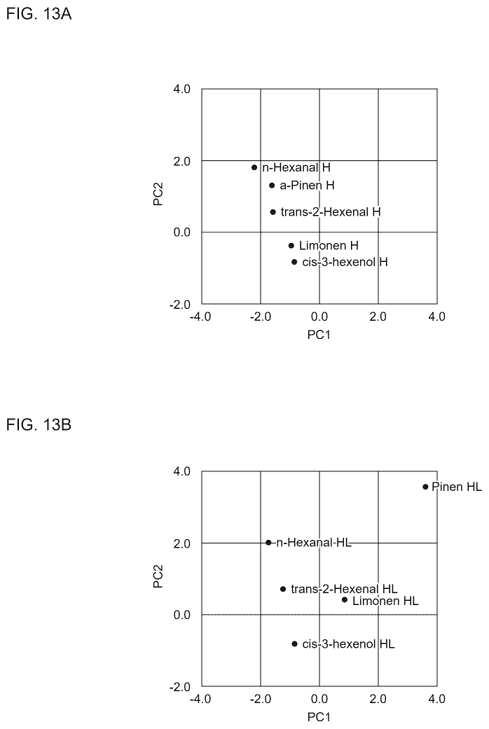

FIG. 11 is a schematic configuration diagram showing a configuration of a measurement apparatus 27A according to a second embodiment of the present technology.

The measurement apparatus 27A according to this embodiment includes a control unit 271A and the memory 271d. The control unit 271A includes the acquisition unit 271a, the calculation unit 271b, an analysis unit 271e, and a determination unit 271c.

Since the difference from the above first embodiment is the determination unit 271c and the analysis unit 271e, the determination unit 271c and the analysis unit 271e will be mainly described. The description of the acquisition unit 271a, the calculation unit 271b, and the memory 271d is simplified or omitted in some cases.

In this embodiment, in the processing procedure for determining the type of gas, a step of performing the main component analysis by the analysis unit 271e on the basis of the calculated sensitivity is executed after Step 109 in the flowchart of FIG. 10. The processing procedure is different from that in the first embodiment in that the type of reducing gas is then determined by the determination unit 271c on the basis of the calculation result calculated by the analysis unit 271e.

Here, the main component analysis will be described. The main component analysis is a type of multivariate analysis, and is a statistical data analysis method for summarizing a large number of variables into fewer variables.

Next, the analysis method of the main component analysis will be described. When performing main component analysis on the data set such as that shown in Table 1, the variance-covariance matrix is obtained from this data first. The variance-covariance matrix is a matrix obtained by, for example, multiplying an n×m matrix of data and an m×n matrix that is the transpose of the data. In this case, the variance-covariance matrix of an n×n matrix is obtained.

Next, an eigenvalue problem is solved for the obtained variance-covariance matrix to acquire the eigenvalue and eigenvector of the data.

Next, a first main component score, a second main component score, . . . are calculated from the obtained eigenvalue and eigenvector, and the respective obtained main component scores are plotted.

The calculation of main component scores will be described. For example, assumption is made that when solving the eigenvalue problem for the variance-covariance matrix of an n×n matrix, the eigenvalues are λ₁, λ₂, . . . , λₙ (λ₁>λ₂> . . . >λₙ), the eigenvector when the eigenvalue is λ₁ is U₁, the eigenvector when the eigenvalue is λ₂ is U₂, . . . , and the eigenvector when the eigenvalue is λₙ is Uₙ. Here, n represents the number of dimensions. At this time, the components are named in order from the largest eigenvalue (largest data variance), as a first main component (PC1), a second main component (PC2), . . . , an n-th main component (PCn).

Here, attention will be paid on the first main component. In the case of the first main component, the eigenvector is U₁, and the respective values of U₁ are parameters of the first main component score. The first main component score is calculated from the parameters and the original data. The second and subsequent main component scores are calculated in the same manner.

The determination unit 271c determines the type of gas on the basis of the results of the main component analysis described above. The method of determining the type of gas is as follows.

FIG. 12 is a diagram plotting main component scores, where Part (A) is a diagram plotting main component scores of gases to be identified, which were calculated in advance, Part (B) is a diagram showing the main component analysis of an unknown gas X and prior data, and Part (c) is a diagram in which data regions of gases to be identified overlap with each other.

As the procedure for determining the type of gas by the main component analysis, a plurality of pieces of sensitivity data of each of odors (an odor 1 and an odor 2) to be identified is calculated first (a plurality of times, different concentrations, etc.) first.

Next, the main component analysis is performed to plot main component scores (Part (A) of FIG. 12). Here, in the case where data regions of the odor 1 and the odor 2 overlap with each other as shown in Part (c) of FIG. 12, the type, number, drive temperature, and the like of the sensors are changed to perform optimization such that the data regions do not overlap. Further, by removing sensors whose reactions do not change depending on the odor, identification is possible with fewer sensors. That is, it is favorable to use a sensor whose eigenvector differs depending on the odor.

Next, the unknown odor X is measured to calculate the sensitivity. The main component analysis is performed with the prior data as shown in Part (A) of FIG. 12 to obtain main component scores. On the basis of the position of the main component score of the unknown odor X and the data regions of the main component scores of the prior data, the determination unit 271c determines which of the odor regions it enters (Part (B) of FIG. 12). In the case of the plot as shown in Part (B) of FIG. 12, the determination unit 271c determines that the unknown odor X is the odor 1. Here, the determination unit 271c may identify the type of gas detected from the above-mentioned memory 271d.

Here, Table 2 shows the main component scores calculated in this embodiment. HL in the table indicates using data at a high temperature and a low temperature.

TABLE 2

| [Main component scores] | PC1 | PC2 | PC3 |
|---|---|---|---|
| Limonen HL | 0.8495651 | 0.435463 | 0.740718 |
| a-Pinen HL | 3.5761527 | 3.589499 | 0.650458 |
| cis-3-hexenol HL | −0.849349 | −0.81288 | 0.441177 |
| n-Hexanal HL | −1.724468 | 2.039597 | −1.19999 |
| trans-2-Hexenal HL | −1.247701 | 0.729339 | −0.60139 |
| Limonen H | −0.980779 | −0.36293 | 1.009515 |
| a-Pinen H | −1.63543 | 1.3135 | 1.450385 |
| cis-3-hexenol H | −0.861582 | −0.81826 | 0.44445 |
| n-Hexanal H | −2.232457 | 1.821035 | −0.97284 |
| trans-2-Hexenal H | −1.601196 | 0.577407 | −0.45855 |
| Limonen L | 1.5886641 | −1.59402 | −0.28119 |
| a-Pinen L | 4.9699031 | −0.11641 | −0.81232 |
| cis-3-hexenol L | −0.229446 | −2.38702 | −0.01566 |
| n-Hexanal L | 0.2663088 | −2.17385 | −0.23954 |
| trans-2-Hexenal L | 0.1118153 | −2.24048 | −0.15523 |

Further, FIG. 13 is a graph plotted on the basis of main component scores, where Part (A) is a diagram plotting only high-temperature data and Part (B) is a diagram plotting high-temperature data and low-temperature data (values of main component scores in Table 2).

As shown in Part (A) of FIG. 13, when the main component analysis is performed with only high-temperature data, the plots are close to each other, making it difficult to perform optimization such that the data regions do not overlap with each other as in Part (A) in FIG. 13. However, when the plots are far apart as in Part (B) of FIG. 13, it is easier to perform optimization such that data regions do not overlap with each other.

As a result, by using not only the sensitivity properties at a high temperature but also the sensitivity properties at a low temperature, it is possible to more easily identify the reducing gas to be detected.

### Modified Example 1

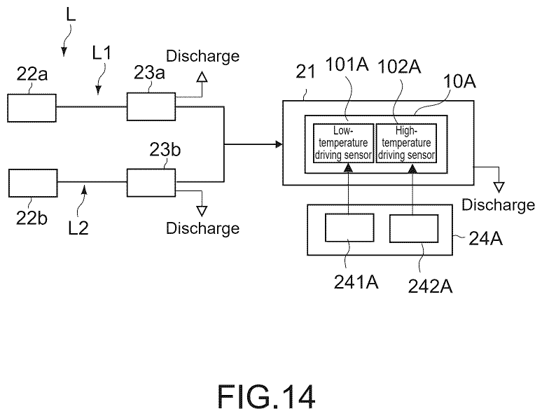

Although the semiconductor sensor 10 is measured at a low temperature and a high temperature in sequence in this example of the present technology as described above, simultaneous measurement may be performed. FIG. 14 is a schematic configuration diagram of the semiconductor sensor 10 according to a modified example 1 that includes a low-temperature driving sensor 101A and a high-temperature driving sensor 102A.

The low-temperature driving sensor 101A and the high-temperature driving sensor 102A are disposed in the measurement chamber 21. The heating unit 24A includes a low-temperature heating unit 241A that heats the low-temperature driving sensor 101A and a high-temperature heating unit 242A that heats the high-temperature driving sensor 102A. As a result, it is possible to simultaneously calculate the change in the resistance value (first sensitivity) of the semiconductor sensor 10 at a low temperature and the change in the resistance value (second sensitivity) of the semiconductor sensor 10 at a high temperature. This allows the type of gas to be determined quickly.

### Modified Example 2

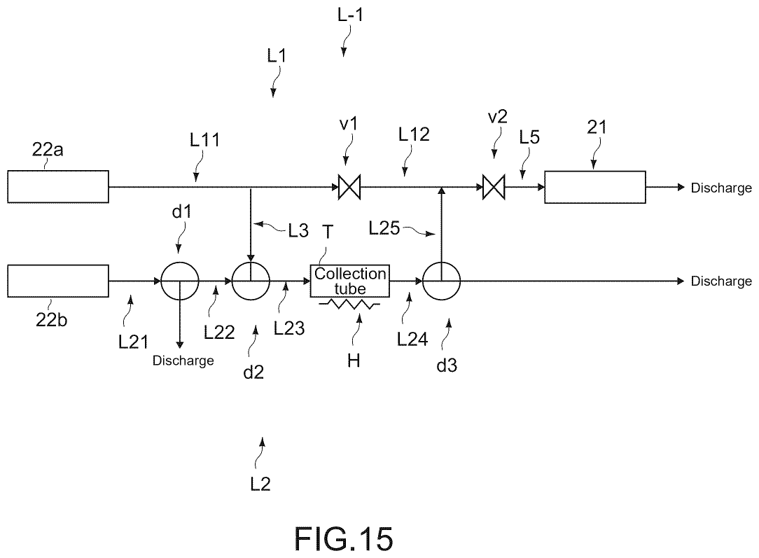

Further, FIG. 15 is a schematic configuration diagram of a gas supply unit L-1 in a modified example 2. Here, although a reducing gas is supplied by only a permeator in the example of the present technology, a collection tube T may be used.

Here, the dry air introduction line L1 includes a line L11 from the first gas control unit 22a to a first valve portion v1 and a line L12 from the first valve portion v1 to a second valve portion v2.

The reducing gas introduction line L2 includes a line L21 from the second gas control unit 22b to a first three-way valve d1, a line L22 from the first three-way valve d1 to a second three-way valve d2, a line L23 from the second three-way valve d2 to the collection tube T, a line L24 from the collection tube T to a third three-way valve d3, and a line L25 that merges to the line L12 of the dry air introduction line L1 from the third three-way valve d3.

The reducing gas introduction line L2 further includes a branch line L3 that branches from the line L11 of the dry air introduction line L to the second three-way valve d2. From the second valve portion v2 to the measurement chamber 21 is a line L5 through which the first gas and the second gas are supplied to the measurement chamber 21.

The collection tube T includes an adsorption material that adsorbs the reducing gas supplied from the second gas control unit 22b and functions also as a supply source that supplies the reducing gas to the measurement chamber 21. The adsorption material is formed of, for example, silica gel, a porous polymer, or activated carbon, and can be desorbed by heating with a heater H or the like. In the case of analyzing a component of a low concentration, it is possible to evaluate the gas with higher sensitivity by concentrating the component in the collection tube T and then supplying the concentrated component to the measurement chamber 21.

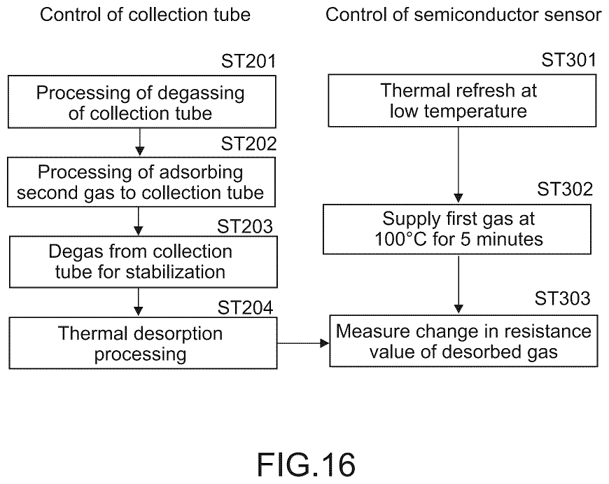

The processing procedure for supplying a gas in this modified example will be shown below. FIG. 16 is a diagram showing a control flow of the collection tube T and a control flow of the semiconductor sensor 10.

The control flow of the collection tube T will be described. First, the second three-way valve d2 is controlled such that the first gas flows into the collection tube T, and the collection tube T is heated by the heater H (Step 201). As a result, it is possible to remove impurity molecules adsorbed to the collection tube T. At this time, the third three-way valve d3 is controlled such that the first gas flows to the exhaust side.

Next, the first three-way valve d1 is controlled such that a second gas flows to the side of the collection tube T, and the second gas is caused to flow into the collection tube T and be adsorbed thereto (Step 202).

Next, the first gas is caused to flow into the collection tube T by controlling the second three-way valve d2, and the first gas that has not collected by the adsorption material and remained in the collection tube T or pipe is removed (Step 203).

While causing the first gas to flow through the collection tube T, the heater H heats the collection tube T that functions as a supply source of a reducing gas to desorb the second gas adsorbed to the collection tube T, and the second gas is caused to flow through the line L24, the line L25, the line L12, the second valve portion v2, and the line L5 in this order to be supplied to the measurement chamber 21. At this time, the first valve portion v1 is closed to prevent the second gas from flowing back.

The control flow of the semiconductor sensor 10 will be described. First, thermal refresh is performed while causing the first gas to flow into the semiconductor sensor 10 (Step 301). At this time, Step 201 may be performed at the same time.

Next, while causing the first gas to flow into the semiconductor sensor 10, the semiconductor sensor 10 is caused to be a set temperature (e.g., 100° C.), and a reference resistance value (R1air) is supplied for 5 minutes (Step 302).

The second gas desorbed by heating from the collection tube T is supplied to the measurement chamber 21, and the change in the resistance is measured (Step 303).

In the case of analyzing a gas component of a low concentration, by measuring the change in the resistance value of the gas by the above flow, it is possible to evaluate the gas with higher sensitivity by concentrating the gas in the collection tube T and then supplying the concentrated gas to the measurement chamber 21.

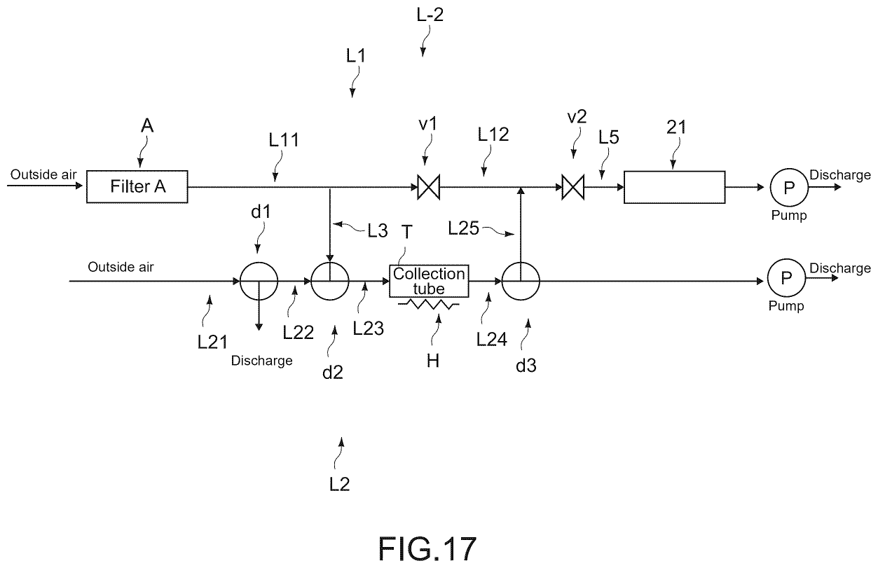

Further, FIG. 17 is a schematic configuration diagram of the gas supply unit L-2 obtained by replacing the first gas control unit 22a in the modified example 2 with a filter A and omitting a permeator. As shown in FIG. 17, the first gas control unit 22a in the modified example 2 may be replaced with the filter A and the permeator may be omitted. The filter A is a filter that removes moisture and impurity molecules contained in the outside air, and, for example, activated carbon, silica gel, or zeolite is used. Further, the gas supplied to the measurement chamber 21 and the gas to be discharged from the third three-way valve d3 to the outside may be discharged by a pump P.

### Modified Example 3

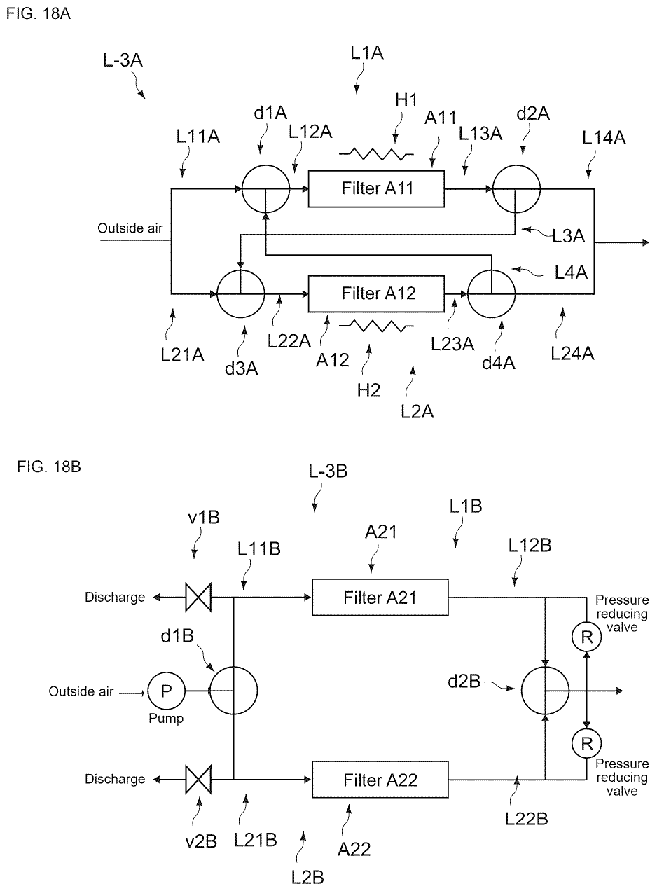

FIG. 18 is shows a modified example of the filter A in FIG. 17, where Part (A) is a schematic configuration diagram showing a gas supply unit L-3A including a regenerative filter that can be regenerated by combining a plurality of filters and is capable of constantly supplying air from which moisture and the like have been removed, and Part (B) is a schematic configuration diagram of a gas supply unit L-3B in which the filter A includes a plurality of filters.

As shown in Part (A) of FIG. 18, the gas supply unit L-3A includes a line L1A as a first passage and a line L2A as a second passage. The line L1A includes a line L11A that connects the outside air and a three-way valve d1A, a line L12A from the three-way valve d1A to a filter A11, a line L13 from the filter A11 to a three-way valve d2A, and a line L14 from the three-way valve d2A to the measurement chamber 21 (not shown). The line L2A includes a line L21A that connects the outside air and a three-way valve d3A, a line L22A from the three-way valve d3A to a filter A12, a line L23 from the filter A12 to a three-way valve d4A, and a line L24 from the three-way valve d4A to the measurement chamber 21 (not shown). Note that the line L11A and the line L21A include a common line on part of the outside air side, and the line L14A and the line L24A include a common line on part of the side of the measurement chamber 21.

Further, the line L1A includes a first switching unit L3A as a drain passage connected from the three-way valve d2A to the three-way valve d3A, and a heater H1 that heats the filter A11. The line L2A includes a second switching unit L4A as a drain passage connected from the three-way valve d4A to the three-way valve d1A, and a heater H2 that heats the filter A12.

The filter A11 and the filter A12 collect moisture, a reducing gas to be detected, and the like. Here, the “collect” does not necessarily mean that the above substances are completely collected by the filter, but means that the filter has a function of collecting the above substances.

In the gas supply unit L-3A, first, the outside air flows into the filter A11, and dry air is supplied to the measurement chamber 21 through the line L14A. At this time, the three-way valve d2A is controlled by the drive control unit 26 (not shown) such that dry air is supplied not to L3A but only to L14A. Further, the three-way valve d3A is controlled by the drive control unit 26 (not shown) such that the outside air does not flow into the filter A12.

Next, in the case where the filter A11 is filled with moisture or the like, it is exhausted to the outside. At this time, the air atmosphere obtained by passing the outside air through the filter A12 is supplied to the filter A11 through the line L14A, and exhausted to the outside by heating the filter A11 by the heater H1. That is, air from which moisture and the like have been removed by the filter A12 on the side where moisture or the like is not contained is supplied to the filter A12 filled with moisture or the like, and exhausted to the outside by heat treatment, allowing the filters to be regenerated and used alternately. Thus, the measurement unit 28 can be used continuously.

As shown in Part (B) of FIG. 18, the gas supply unit L-3B is a heatless air dryer. The heatless air dryer utilizes the property of a desiccant to constantly maintain equilibrium with the water vapor concentration of the air around it. The heatless air dryer includes two cylinders (filters), alternately repeats an adsorption process in which the desiccant adsorbs water vapor in the moist air and a regeneration process in which the desiccant releases moisture due to dry air, constantly dries the moist air that has entered the apparatus and supplies the dried air from the outlet of the apparatus.

The gas supply unit L-3B includes a line L1B and a line L2B. The line L1B includes a line L11B from a three-way valve d1B to a filter A21, a line L12B from the filter A21 to a three-way valve d2B, and a valve unit v1B that controls the exhaust side of L11B.

The line L2B includes a line L21B from the three-way valve d1B to a filter A22, a line L22B from the filter A22 to the three-way valve d2B, and a valve unit v2B that controls the exhaust side of L21B.

In the gas supply unit L-3B, first, moisture and a reducing gas of the outside air pass through the line L11B from the three-way valve d1B and are collected in the filter A21. At this time, the three-way valve d1B is controlled such that the outside air does not flow into the filter A22. Next, they pass through the three-way valve d2B from the filter A21 and are supplied to the measurement chamber 21 (not shown). At this time, part of the air that has passed through the three-way valve d2B is reduced in pressure to the atmospheric pressure through a pressure reducing valve R, and enters the filter A21, where moisture thereof is removed by the desiccant of the filter A21. It is emitted to the outside (atmosphere) via the valve unit v1B.

The air that has been pressurized by the pump and passed through the filter A22 is reduced in pressure by the pressure reducing valve, which makes it possible to further increase the degree of dryness and improve the regeneration efficiency of the filter A21. The same applies also when the outside air flows into the filter A21 via the three-way valve d1B.

### Modified Example 4

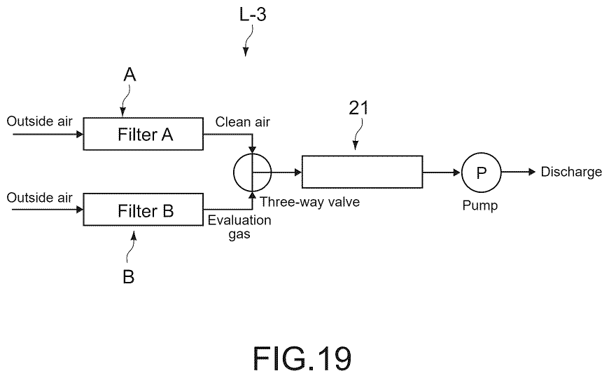

FIG. 19 is a schematic configuration diagram of a gas supply unit L-3 that supplies the first gas and the second gas to the measurement chamber 21 through the filter A and a filter B, respectively. The filter A is a filter that removes moisture and volatile organic molecules contained in the outside air, and is, for example, activated carbon, silica gel, zeolite, or a hollow fiber film. The filter B is a filter using silica gel, zeolite, a hollow fiber film, or the like, which is easy to collect moisture in order to remove moisture. The filter B is not necessarily required. The pump P for discharging gases is installed behind the measurement chamber 21.

### Other Modified Examples

Although the type of gas is identified by the main component analysis in the example of the present technology as described above, the present technology is not limited thereto, and the type of gas may be identified by various methods such as support vector machines, decision trees, random forests, and Deep Learning.

Although the hydrocarbon substances emitted from plants are described as an example in the example of the present technology, the present technology is not limited thereto, and, for example, hydrocarbon substances emitted from animals or humans may be detected by the MOx sensor according to the present technology. The hydrocarbon substances emitted from animals and humans are, for example, nonanal, and lung cancer can be diagnosed by detecting it by the MOx sensor.

### Application Example

Although hydrocarbon substance emitted from plants are detected by only the MOx sensor or in combination with a filter in the example of the present technology, the present technology is not limited thereto. Hydrocarbon substances emitted from plants may be adsorbed, concentrated, and separated using gas chromatography, molecular sieves (zeolite/carbon molecular sieves), or molecular template polymers, and then detected by the MOx sensor according to the present technology. As a result, it is possible to improve the detection accuracy of the MOx sensor according to the present technology.

Note that the present technology may also take the following configurations.

(1) A measurement apparatus, including:
a measurement unit that includes one or more semiconductor sensors each including an adsorption layer formed of metal oxide, a measurement chamber that houses the semiconductor sensor, and a heating unit that heats the semiconductor sensor to a first temperature, a second temperature, or a third temperature, the second temperature and the third temperature being higher than the first temperature;
a gas supply unit that includes a first gas supply line and a second gas supply line, the first gas supply line supplying a first gas that does not contain a reducing gas to be detected to the measurement chamber, the second gas supply line supplying a second gas that contains the reducing gas to the measurement chamber; and
a control unit that controls the measurement unit and the gas supply unit,
the control unit being capable of executing
   refresh treatment for cleaning the adsorption layer while heating the semiconductor sensor to the third temperature in an atmosphere of the first gas,
   low-temperature measurement treatment for measuring a resistance value of the semiconductor sensor while heating the semiconductor sensor to the first temperature in an atmosphere of the second gas, and
   high-temperature measurement treatment for measuring the resistance value of the semiconductor sensor while heating the semiconductor sensor to the second temperature in the atmosphere of the second gas,
the control unit executing the low-temperature measurement treatment as a process following the refresh treatment.
   (2) The measurement apparatus according to (1) above, in which
the control unit executes
   low-temperature measurement pre-treatment for measuring the resistance value of the semiconductor sensor while heating the semiconductor sensor to the first temperature in the atmosphere of the first gas as a process between the refresh treatment and the low-temperature measurement treatment, and
   high-temperature measurement pre-treatment for measuring the resistance value of the semiconductor sensor while heating the semiconductor sensor to the second temperature in the atmosphere of the first gas as a process before the high-temperature measurement treatment,
and calculates
   a first sensitivity that is a ratio of the resistance value measured in the low-temperature measurement pre-treatment to the resistance value measured in the low-temperature measurement treatment, and
   a second sensitivity that is a ratio of the resistance value measured in the high-temperature measurement pre-treatment to the resistance value measured in the high-temperature measurement treatment.
      (3) The measurement apparatus according to (1) or (2) above, in which
the first temperature is 25° C. or more and 200° C. or less, and
the second temperature is 200° C. or more and 500° C. or less.
   (4) The measurement apparatus according to any one of (1) to (3) above, in which
the third temperature is 300° C. or more and 600° C. or less.
   (5) The measurement apparatus according to any one of (1) to (4) above, in which
an air atmosphere in which the reducing gas is not present is dry air with a relative humidity of 10% or less.
   (6) The measurement apparatus according to any one of (1) to (5) above, in which
the semiconductor sensor includes a first semiconductor sensor for the low-temperature measurement treatment and a second semiconductor sensor for the high-temperature measurement treatment, and
the control unit simultaneously executes the low-temperature measurement treatment and the high-temperature measurement treatment.
   (7) The measurement apparatus according to any one of (1) to (5) above, in which
the second gas supply line includes a branch line that branches from the first gas supply line and a supply source of the reducing gas disposed in the branch line, and supplies the second gas to the measurement chamber as a mixed gas of the first gas and the reducing gas.
   (8) The measurement apparatus according to any one of (1) to (7) above, in which
the first gas supply line includes a first passage that includes a first filter for collecting moisture and the reducing gas, a second passage that includes a second filter for collecting the moisture and the reducing gas, and a switching unit that selectively switches between the first passage and the second passage.
   (9) The measurement apparatus according to any one of (1) to (8) above, in which
the first gas supply line further includes a heater that is capable of heating the first filter and an exhaust passage for discharging moisture desorbed from the first filter by the heater.
   (10) The measurement apparatus according to (2) above, in which
the control unit includes a determination unit that determines a type of the reducing gas on a basis of the first sensitivity and the second sensitivity.
   (11) The measurement apparatus according to (10) above, in which
the determination unit determines whether or not the reducing gas contains a hydrocarbon substance emitted from a living thing such as a plant, an animal, and a human.
   (12) The measurement apparatus according to (11) above, in which
the hydrocarbon substance is a substance that contains a terpene organic compound.
   (13) A measurement method, including:
controlling, by a control unit,
   a measurement unit that includes one or more semiconductor sensors each including an adsorption layer formed of metal oxide, a measurement chamber that houses the semiconductor sensor, and a heating unit that heats the semiconductor sensor to a first temperature, a second temperature, or a third temperature, the second temperature and the third temperature being higher than the first temperature, and a gas supply unit that includes a first gas supply line and a second gas supply line, the first gas supply line supplying a first gas that does not contain a reducing gas to be detected to the measurement chamber, the second gas supply line supplying a second gas that contains the reducing gas to the measurement chamber;
executing, by the control unit,
   refresh treatment for cleaning the adsorption layer while heating the semiconductor sensor to the third temperature in an atmosphere of the first gas,
   low-temperature measurement treatment for measuring a resistance value of the semiconductor sensor while heating the semiconductor sensor to the first temperature in an atmosphere of the second gas, and
   high-temperature measurement treatment for measuring the resistance value of the semiconductor sensor while heating the semiconductor sensor to the second temperature in the atmosphere of the second gas; and
executing, by the control unit, the low-temperature measurement treatment as a process following the refresh treatment.

### REFERENCE SIGNS LIST

10 semiconductor sensor
11 substrate
12a, 12b electrode
13 adsorption layer
14 heating layer
20 measurement system
25 control unit
26 drive control unit
27 measurement apparatus
271a acquisition unit
271b calculation unit
271c determination unit
271e analysis unit
28 measurement unit
L1 first gas supply line
L2 second gas supply line

1. A measurement apparatus, comprising:
a measurement unit that includes one or more semiconductor sensors each including an adsorption layer formed of metal oxide, a measurement chamber that houses the semiconductor sensor, and a heating unit that heats the semiconductor sensor to a first temperature, a second temperature, or a third temperature, the second temperature and the third temperature being higher than the first temperature;
a gas supply unit that includes a first gas supply line and a second gas supply line, the first gas supply line supplying a first gas that does not contain a reducing gas to be detected to the measurement chamber, the second gas supply line supplying a second gas that contains the reducing gas to the measurement chamber; and
a control unit that controls the measurement unit and the gas supply unit,
the control unit being capable of executing
refresh treatment for cleaning the adsorption layer while heating the semiconductor sensor to the third temperature in an atmosphere of the first gas,
low-temperature measurement treatment for measuring a resistance value of the semiconductor sensor while heating the semiconductor sensor to the first temperature in an atmosphere of the second gas, and
high-temperature measurement treatment for measuring the resistance value of the semiconductor sensor while heating the semiconductor sensor to the second temperature in the atmosphere of the second gas,
the control unit executing the low-temperature measurement treatment as a process following the refresh treatment.

2. The measurement apparatus according to claim 1, wherein
the control unit executes
low-temperature measurement pre-treatment for measuring the resistance value of the semiconductor sensor while heating the semiconductor sensor to the first temperature in the atmosphere of the first gas as a process between the refresh treatment and the low-temperature measurement treatment, and
high-temperature measurement pre-treatment for measuring the resistance value of the semiconductor sensor while heating the semiconductor sensor to the second temperature in the atmosphere of the first gas as a process before the high-temperature measurement treatment,
and calculates
a first sensitivity that is a ratio of the resistance value measured in the low-temperature measurement pre-treatment to the resistance value measured in the low-temperature measurement treatment, and
a second sensitivity that is a ratio of the resistance value measured in the high-temperature measurement pre-treatment to the resistance value measured in the high-temperature measurement treatment.

3. The measurement apparatus according to claim 1, wherein
the first temperature is 25° C. or more and 200° C. or less, and
the second temperature is 200° C. or more and 500° C. or less.

4. The measurement apparatus according to claim 1, wherein
the third temperature is 300° C. or more and 600° C. or less.

5. The measurement apparatus according to claim 1, wherein
an air atmosphere in which the reducing gas is not present is dry air with a relative humidity of 10% or less.

6. The measurement apparatus according to claim 1, wherein
the semiconductor sensor includes a first semiconductor sensor for the low-temperature measurement treatment and a second semiconductor sensor for the high-temperature measurement treatment, and
the control unit simultaneously executes the low-temperature measurement treatment and the high-temperature measurement treatment.

7. The measurement apparatus according to claim 1, wherein
the second gas supply line includes a branch line that branches from the first gas supply line and a supply source of the reducing gas disposed in the branch line, and supplies the second gas to the measurement chamber as a mixed gas of the first gas and the reducing gas.

8. The measurement apparatus according to claim 1, wherein
the first gas supply line includes a first passage that includes a first filter for collecting moisture and the reducing gas, a second passage that includes a second filter for collecting the moisture and the reducing gas, and a switching unit that selectively switches between the first passage and the second passage.

9. The measurement apparatus according to claim 8, wherein
the first gas supply line further includes a heater that is capable of heating the first filter and an exhaust passage for discharging moisture desorbed from the first filter by the heater.

10. The measurement apparatus according to claim 2, wherein
the control unit includes a determination unit that determines a type of the reducing gas on a basis of the first sensitivity and the second sensitivity.

11. The measurement apparatus according to claim 9, wherein
the determination unit determines whether or not the reducing gas contains a hydrocarbon substance emitted from a living thing such as a plant, an animal, and a human.

12. The measurement apparatus according to claim 11, wherein
the hydrocarbon substance is a substance that contains a terpene organic compound.

13. A measurement method, comprising:
controlling, by a control unit,
a measurement unit that includes one or more semiconductor sensors each including an adsorption layer formed of metal oxide, a measurement chamber that houses the semiconductor sensor, and a heating unit that heats the semiconductor sensor to a first temperature, a second temperature, or a third temperature, the second temperature and the third temperature being higher than the first temperature, and a gas supply unit that includes a first gas supply line and a second gas supply line, the first gas supply line supplying a first gas that does not contain a reducing gas to be detected to the measurement chamber, the second gas supply line supplying a second gas that contains the reducing gas to the measurement chamber;
executing, by the control unit,
refresh treatment for cleaning the adsorption layer while heating the semiconductor sensor to the third temperature in an atmosphere of the first gas,
low-temperature measurement treatment for measuring a resistance value of the semiconductor sensor while heating the semiconductor sensor to the first temperature in an atmosphere of the second gas, and
high-temperature measurement treatment for measuring the resistance value of the semiconductor sensor while heating the semiconductor sensor to the second temperature in the atmosphere of the second gas; and
executing, by the control unit, the low-temperature measurement treatment as a process following the refresh treatment.
